(19) United States
(12) Patent Application Publication
(10) Pub. No.: US 2025/0009240 A1
(43) Pub. Date: Jan. 9, 2025

(54) MEASUREMENT OF PARAMETERS RELATED TO THE HEALTH OF A USER

(71) Applicant: LMD IP, LLC, Oklahoma City, OK (US)

(72) Inventors: Christopher Elliott, St-Sulpice (CH); Mark-Eric Jones, Penthalaz (CH)

(73) Assignees: LMD IP, LLC, Oklahoma City, OK (US); LEMAN MICRO DEVICES SA, Lausanne (CH); LEMAN MICRO DEVICES, LLC, Oklahoma City, OK (US)

(21) Appl. No.: 18/711,565

(22) PCT Filed: Nov. 18, 2022
(86) PCT No.: PCT/EP2022/082482

§ 371 Date: May 17, 2024

(30) Foreign Application Priority Data
Nov. 19, 2021 (GB) 2116701.0

(51) Int. Cl. A61B 5/022; A61B 5/00; A61B 5/01; A61B 5/021; A61B 5/0225; A61B 5/0235

(52) U.S. Cl. CPC A61B 5/02241; A61B 5/01; A61B 5/02141; A61B 5/0225; A61B 5/0235; A61B 5/489; A61B 5/6816; A61B 5/6826; A61B 5/7271; A61B 2560/045; A61B 2560/0462; A61B 2562/0247; A61B 2562/0271

(57) ABSTRACT

A device is disclosed for measuring blood pressure comprising a signal acquisition component comprising a flexible and essentially incompressible gel or resin, a surface of which is adapted in use to contact a body part; a pressure sensor embedded in the gel or resin, one or more photoemitters for emitting light into the body part when the body part is in contact with the surface of the gel or resin, one or more photodetectors for detecting light scattered by or transmitted through the body part; a processing means which is programmed for analysing the signals from the pressure sensor and the photodetector(s) to provide a measurement of blood pressure in the body part; and a mechanism for applying force, which is adapted to apply varied force to the body part in a direction towards the surface of the gel or resin.

The present invention relates to a device and method for the non-invasive measurement of blood pressure and associated health-related parameters.

### BACKGROUND

Leman Micro Devices has invented a unique cuff-less occlusion device for the measurement of blood pressure. This is referred to herein as “the Leman device”. The Leman device is the subject of the patent applications summarised below. The Leman device uses the traditional Riva-Rocci principle of applying pressure to a body part to balance the pressure in the artery but, unlike in the use of a conventional cuff, the Leman device is programmed so that, in use, it asks the user to press harder or softer to achieve a desired range of pressures. The Leman device is programmed with algorithms which are adapted to analyse pressure and occlusion data received in any order. The pressure does not need to be continuously increased or continuously decreased.

WO2013/001265 discloses a Personal Hand-Held Monitor (PHHM) in which a signal acquisition device (SAD) is integrated with a Personal Hand-Held Computing Device (PHHCD), such as a cell phone, and is adapted for the measurement of, for example, blood pressure or one or more of several other health-related parameters. The SAD is adapted to be pressed against a body part or to have a body part pressed against it. The body part may be, for example, the side of a finger. This permits a cuff-less occlusion measurement.

WO2014/125431 discloses several improvements of the PHHM described in WO2013/001265, including the use of: a gel to measure pressure; a saddle-shape surface to interact with a body part; corrections for the actual position of an artery relative to the monitor; and the use of interactive instructions to the user.

WO2014/125355 discloses improvements of the PHHM disclosed in WO2013/001265 including improvements to the specificity and accuracy of the measurements.

WO2016/096919 discloses several further improvements of the PHHMs described in WO2013/001265 and WO2014/125431, including: improvements to the gel and pressure sensing means; the use of mathematical procedures for extracting blood pressures; the use of improved signal processing systems; a means for identifying the user; improvements to the electrical systems for measurement; and several aspects of test and calibration of the PHHM.

WO2017/140748 discloses further improvements to a PHHM for extracting blood pressure and several other health-related parameters that can be derived from the measured data.

WO2017/198981 discloses improvements to the PHHM disclosed in WO2014/125355 whereby the PHHM can be built using small and inexpensive components.

WO2019/211807 discloses several improvements to the PHHMs disclosed above, including aspects where the PHHM is adapted to use the fingertip or an artery in the cheek, both of which result in increased variation in applied pressure when compared to the PHHMs disclosed in the earlier applications.

WO2021/124071 discloses certain discoveries regarding new capabilities of the devices disclosed by the earlier inventions and a further improvement in the signal processing.

WO2013/001265, WO2014/125355, WO2014/125431, WO2016/096919, WO2017/140748, WO2017/198981, WO2019/211807 and WO2021/124071 are all in the name of Leman Micro Devices SA and are therefore collectively referred to as “the Leman applications”. The Leman applications are hereby incorporated into the present application in their entirety by reference.

The PHHMs disclosed in the Leman applications are effective, accurate, easy to use and may be integrated into a cell phone. Cell phones are manufactured in quantities of hundreds of millions and the price of their components is critical. The PHHMs disclosed in the Leman applications allow the cost of blood pressure measurement to be reduced to a level that is acceptable for a cell phone, in part because they eliminate the expensive and heavy components of conventional devices that ensure a constant applied pressure.

However, some users find it difficult to control the force with which the PHHM is pressed against the body part or the body part is pressed against the PHHM. The device of the present invention allows the use of the same inexpensive and readily available measurement technique without the need for accurate control.

The device and the method of the present invention rely on the principles disclosed in the Leman applications. In particular, the device of the present invention includes a signal acquisition component which is adapted to acquire signals related to the flow of blood through a body part and the pressure in the body part. The blood flow and pressure signals produced by the signal acquisition component of the device of the present invention can be processed, in whatever order the signals are received, to produce a measurement of blood pressure. The disclosures of the Leman applications in relation to these aspects are particularly relevant to the device of the present invention.

In addition, it is desirable to have a device, such as that of the previous Leman applications, that can provide an estimate for a user's core body temperature. When doctors refer to a patient's temperature, they typically mean the core temperature. In practice, this is taken to mean the temperature of a blood vessel such as arterial blood, ideally at the aorta but if not at a location where the blood will not have changed temperature significantly.

The temperature of the arterial blood is hard to measure non-invasively. Quasi non-invasive techniques, such as measurement of the temperature of the tympanic membrane, give an approximation but it would be easier and more useful if the measurement could be made on the surface of the skin. Many commercial thermometers measure skin temperature, either by a contact thermometer or by detecting infra-red radiation from the skin, but all would benefit from greater accuracy.

### SUMMARY OF THE INVENTION

It has been discovered that an occlusion device which includes a mechanism for applying a force to a body part is easier to use than a PHHM of the type disclosed in the Leman applications.

A first aspect of the present invention provides a device for the measurement of blood pressure in a body part comprising:
a signal acquisition component comprising:
a flexible and essentially incompressible gel or resin, one surface of which is flat and is adapted in use to contact a body part; and
a pressure sensor embedded in the gel or resin;
one or more photoemitters for emitting light into the body part when the body part is in contact with the flat surface;
one or more photodetectors for detecting light scattered by or transmitted through the body part;
a collection means for collecting signals from the pressure sensor and the photodetector(s);
a communication means for communicating the signals collected by the collection means to a processing means which is programmed with algorithms for analysing the signals from the pressure sensor and the photodetector(s) to provide a measurement of blood pressure in the body part; and
a mechanism for applying, when the body part is in contact with the flat surface, a varying force to the body part in a direction towards the flat surface.

The mechanism may be arranged such that the force is applied directly to the body part. Alternatively, the force may be applied indirectly to the body part through a hinged mechanism or a lever. A soft pad may be provided to be located, in use, between the mechanism and the body part to ameliorate any discomfort that the use of the device may cause.

In use of the device of the present invention, the mechanism is used to create a range of forces on one side of the body part. The instantaneous pressures that that force causes inside the body part are measured by the pressure sensor and the flow of blood through the arteries of the body part is detected by the photodetector(s). Since the mechanism provides, in use, a varying force on the body part, a range of pressures is created in the body part. The range of pressures is measured by the pressure sensor. The flow of blood through the body part is measured using the photodetector(s). The signals produced by the pressure sensor and the photodetector(s) are then processed as described in the Leman applications to provide a measure of the blood pressure in the body part.

Preferably, the collection means is adapted to digitise the signals from the pressure sensor and the photodetector(s).

The collection means and the communication means may be separate components or parts of a single component, such as an Application Specific Integrated Circuit (ASIC).

The device of the present invention may be adapted to operate with a finger as the body part. Alternatively, the device may be adapted to operate with an earlobe as the body part.

The mechanism may include:
a spring with a liquid or air damper;
a solenoid, an electric motor or a clockwork mechanism;
a pneumatic pump with a bellows, a piston and cylinder or a cuff.

The action of the mechanism may be uncontrolled, in that it may be adapted to generate force which increases or decreases monotonically after the mechanism is activated by the user.

In an alternative, the action of the mechanism may be semi-controlled, in that it may be adapted to generate force which increases or decreases monotonically after the mechanism is activated by the user but the rate of change is controlled by the processing means.

In a further alternative, the action of the mechanism may be controlled, in that it may be adapted to generate force according to a programme determined by the processing means.

It is important to recognise that, in operation of a device according to the invention, it is not necessary for the instantaneous pressure on the body part to increase or decrease monotonically because the device can be adapted to use the actual measured pressure through the skin of the body part and not the force generated by the mechanism to determine the current pressure experienced by the artery. The actions of muscles in the body part may also cause pressure on the pressure sensor so the actual measured pressure might not change monotonically even if the displacement of the mechanism is monotonic.

An embodiment of an uncontrolled mechanism is a spring that is compressed by the user and which then expands at a rate determined by a dash pot or other damping means. The damping means may ensure that the movement of the spring takes around 1 minute so as to allow sufficient time for a full range of pressures, reaching from below Diastolic Blood Pressure to above Systolic Blood Pressure, to be experienced by the body part. Preferably, the damping means uses an oil being forced through a small aperture. Alternatively, the damping means may employ air damping using a spinning vane that is highly geared and so spins very quickly as the mechanism advances.

This embodiment may be simple and inexpensive but the rate of change of the force will depend on the exact properties of the damping means. Both oil damping and air damping depend on temperature and so the rate can change significantly. A second embodiment, illustrating semi-controlled force, allows the processing means to regulate the rate of change, for example by operating a needle valve that determines the rate of flow of the oil. This may have a setting at which the rate goes to zero, allowing the processing means to stop the change of force if it is necessary to repeat measurements at approximately the same pressure.

A third embodiment that provides controlled force uses a solenoid to create the force, where the current through the solenoid is set by the processing means. Typical solenoids that would create the necessary force of around 5 N for a typical finger body part have limited travel and so this embodiment might also include a ratchet mechanism to bring a soft pad firmly onto the body part before starting the forcing and a lever to magnify the force. Alternatively, the third embodiment could use a geared electric motor or clockwork mechanism that is “wound” or preloaded by the user. This is more complex but provides the necessary force and travel with less power than a solenoid.

A further embodiment that provides controlled force uses a pneumatic pump and a bellows, a piston and cylinder or a cuff surrounding the body part and device, to create the force. The action of the pump and of a release valve may be controlled by the processing means to determine the applied force and hence the pressure on the body part. Note that there is no requirement for the pressure generated by the pneumatic means to be the same as the pressure within the body part since the latter is measured directly by the pressure sensor. This property allows a cuff to be used to apply the force without errors arising because the relationship between the pressure in the cuff and the pressure experienced by the arteries in the body part might change between users and during the course of a measurement by one user.

It will be apparent to a person skilled in the art that other embodiments of the mechanism are possible.

The force applied by the mechanism generates pressure in the tissue of the body part. The generated pressure will depend on the size of the body part, the way in which it is placed and on any muscle action or other changes during the measurement. The operation of the device of the present invention is not affected by this because the device measures the instantaneous pressure in the body part, as described in the Leman applications, and the processing means is adapted to use this measured pressure to determine blood pressures, even if the instantaneous pressures do not vary monotonically.

The present invention also provides a system for measuring blood pressure which comprises a device according to the present invention and the processing means.

Preferably, the processing means is adapted to issue instructions to the user.

The processing means may also be adapted to control the photoemitters and/or other components of the device of the present invention.

The processing means may further be adapted to control the variation of the force applied, in use, to the body part by the mechanism. For instance, the force may be varied monotonically upwards or downwards or may be varied randomly.

The processing means may be a separate item and may be connected wirelessly, for instance by Bluetooth or WiFi, or via a cable, such as an I2C or USB cable, to the communication means. In this case, the processing means may be a computer, mobile phone or tablet computer.

Alternatively, the processing means may be incorporated into the device, for instance as part of an ASIC, preferably together with the collection means and the communication means.

A second aspect of the invention relates to a processor for a personal health monitor (PHM), the PHM for determining a parameter related to the health of a user, the processor configured to receive a first temperature measurement from a first temperature sensor associated with a first location on the surface of a body of the user and a second temperature measurement from a second temperature sensor associated with a second location on the surface of the body; wherein the first location and the second location are different. The processor is further configured to determine a first estimate of a core body temperature of the user based on the first temperature measurement, determine an adjustment to the first estimate based on the first temperature measurement and the second temperature measurement and determine the parameter related to the health of the user based upon a second estimate of the core body temperature of the user, the second estimate determined by applying the adjustment to the first estimate of the core body temperature of the user.

In one embodiment the first location may be more proximate to a blood vessel and the second location may be less proximate to the blood vessel, wherein the blood vessel is a vein or artery.

In one embodiment the processor may be configured to determine the first estimate of the core body temperature by identifying which temperature measurement of the first temperature measurement and the second temperature measurement indicates either the largest value of the core body temperature or the largest temperature measurement.

In one embodiment the processor is configured to determine the first estimate of the core body temperature by fitting a function to the first temperature measurement and the second temperature measurement; and optionally wherein the first estimate of the core body temperature is the maximum value of the function.

In one embodiment the adjustment compensates for the difference between the temperature of a skin of the user and the core body temperature.

In one embodiment the adjustment is based on determining a difference between the first temperature measurement and the second temperature measurement.

In a further aspect of the invention, a processor for a personal health monitor (PHM) is disclosed, the PHM for determining a parameter related to the health of a user or assisting in the determination of a parameter related to the health of a user. The processor is configured to receive a first temperature measurement from a first temperature sensor associated with a first location on the surface of a body of the user and a second temperature measurement from a second temperature sensor associated with a second location on the surface of the body wherein the first location and the second location are different. The processor is further configured to determine a location of a blood vessel based on the first temperature measurement and the second temperature measurement and determine an adjustment to a first estimate of a blood pressure of the user based on the location of the blood vessel.

In one embodiment the processor may be further configured to determine the parameter related to the health of the user based upon a second estimate of the blood pressure of the user, the second estimate determined by applying the adjustment to the first estimate of the blood pressure of the user.

In one embodiment the location of the blood vessel is determined by fitting a function to the first temperature measurement and the second temperature measurement; and optionally wherein the location of the blood vessel is where the function is determined to be at a maximum.

In one embodiment the processor is further configured to receive a third temperature measurement from a third temperature sensor associated with a third location on the surface of a body of the user, the third location being different from the first location and the second location, and wherein the function is a Gaussian function.

In one embodiment the function is a Gaussian function.

In one embodiment the adjustment is based on a characteristic of the function.

In one embodiment the function is a Gaussian function, and the characteristic is the variance of the Gaussian function.

In one embodiment the first location and second location are located on a cheek of the user, and the blood vessel is a carotid artery. In another embodiment the first location and second location are located on a forehead of the user and the blood vessel is the temporal artery.

In one embodiment the distance between the two locations may be less than 30 cm, less than 15 cm, less than 5 cm, less than 2 cm, less than 1 cm.

In one embodiment the processor may be configured to output the parameter related to the health of the user.

In one embodiment a personal health monitor (PHM) for determining a parameter related to the health of a user may be provided. The PHM comprising the processor and a signal acquisition device (SAD) comprising a first temperature sensor and a second temperature sensor.

In one embodiment the SAD comprises a housing, wherein the first temperature sensor and the second temperature sensor are fixed within the housing such that movement of the housing causes movement of the first temperature sensor and the second temperature sensor simultaneously.

In one embodiment the housing comprises a contact surface and is configured to be pressed against the first and second locations.

In one embodiment the contact surface is configured to adapt, conform or match a shape of the body part.

In one embodiment the SAD may be disposed in a garment or wearable, and optionally, wherein the garment is any one of a hat, a helmet, a cap, a piece of safety equipment, a headphone or pair of headphones, an earphone, or a virtual reality headset.

In one embodiment the PHM is hand-held.

In another aspect of the invention there is provided a method of determining a parameter related to the health of a user, the method comprising receiving a first temperature measurement from a first temperature sensor associated with a first location on the surface of a body of the user and a second temperature measurement from a second temperature sensor associated with a second location on the surface of the body; wherein the first location and the second location are different. The method further comprises determining a first estimate of a core body temperature of the user based on the first temperature measurement; determining an adjustment to the first estimate based on the first temperature measurement and the second temperature measurement; and determining the parameter related to the health of the user based upon a second estimate of the core body temperature of the user, the second estimate determined by applying the adjustment to the first estimate of the core body temperature of the user.

In another aspect of the invention, there is provided a method of determining a parameter related to the health of a user or assisting in the determination of a parameter related to the health of a user. The method comprises receiving a first temperature measurement from a first temperature sensor associated with a first location on the surface of a body of the user and a second temperature measurement from a second temperature sensor associated with a second location on the surface of the body; wherein the first location and the second location are different. The method further comprises determining a location of a blood vessel based on the first temperature measurement and the second temperature measurement; and determining an adjustment to a first estimate of a blood pressure of the user based on the location of the blood vessel.

In one embodiment a computer-readable medium storing instructions is provided that, when executed by a processor, cause the processor to execute the methods disclosed herein.

The present invention is now described, by way of example only, with reference to the accompanying drawings in which:

### BRIEF DESCRIPTION OF DRAWINGS

FIG. 1 is a cross-section of a signal acquisition component for use in a device according to the present invention;

FIG. 2 is a representation of a first device according to the present invention;

FIG. 3 shows a simple (3a) and a more complex (3b) means for creating a continuously varying force.

FIG. 4 is a representation of a second device according to the present invention in the form of a clip; and

FIG. 5 is a representation of a third device according to the present invention.

FIG. 6 is flow chart showing an exemplary method of determining the core body temperature of the user.

FIG. 7 is a flow chart showing an exemplary method of determining an adjustment to a blood pressure measurement.

FIG. 8a is a top-down view of a first array of sensors that may be used in accordance with the invention.

FIG. 8b is a top-down view of a second array of sensors that may be used in accordance with the invention.

FIG. 9 is a profile view of the anatomy of the human head showing the location of the external carotid artery.

FIG. 10a is a plot showing an exemplary set of temperature measurements that may be received in by an array of temperature sensors disposed over a region of the body comprising a blood vessel.

FIG. 10b is a plot showing the set of temperature measurements of FIG. 10a, further showing a function fit to the temperature measurements in accordance with the invention.

The invention is not limited to the embodiments shown in the accompanying drawings. The scope of the invention is defined in the accompanying claims.

### DETAILED DESCRIPTION

### 1. First Aspect

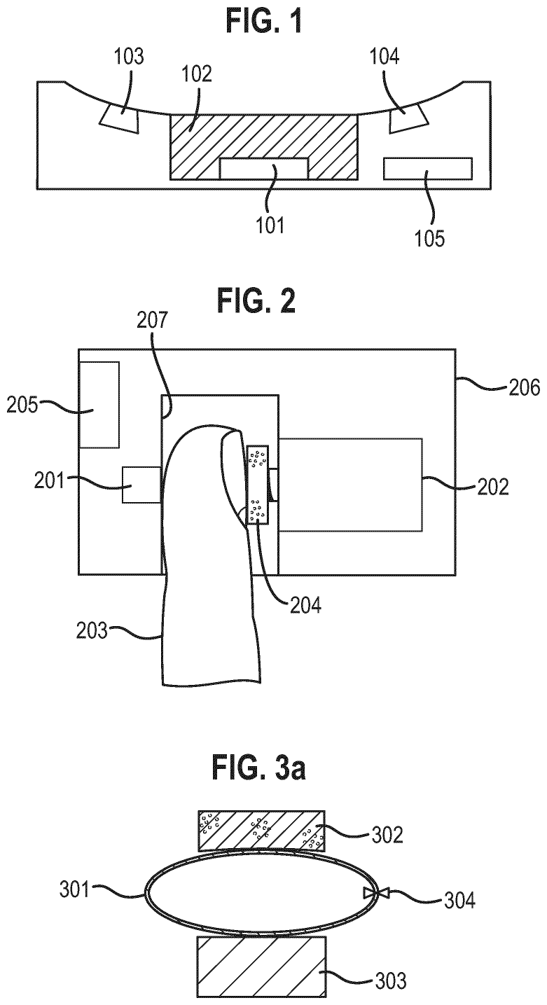

The signal acquisition component shown in FIG. 1, for use in devices of the present invention, comprises a pressure sensor 101 embedded in a flexible and essentially incompressible gel or resin 102 wherein the surface of the gel or resin that, in use, is in contact with the body part is flat. The component also includes a plurality of photoemitters 103 which are arranged to emit light into the body part and a plurality of photodetectors 104 that are arranged to detect light from the photoemitters scattered by or transmitted through the body part.

The component also includes a collection and communicating means 105 in the form of an Application Specific Integrated Circuit (ASIC). This ASIC is adapted to collect and digitise the signals from the pressure sensor 101 and the photodetectors 104 and determine the amount of light emitted by each of the photoemitters 103. It thus serves the function of the collection means. It is also adapted to communicate with a processing means (not shown). It thus also serves the function of the communicating means, either by itself or with one or more additional components. The processing means is programmed with algorithms for processing the signals received by the collection means to provide a value for the blood pressure in the body part in contact with the signal acquisition component 102.

FIG. 2 shows a first device according to the invention. The components of the device are mounted on or in a housing 206 which includes a U-shaped cut-out 207. The device includes a signal acquisition component 201 which includes the photoemitters 103, photodetectors 104, the gel or resin 102 and the pressure sensor 101 although these are not shown in FIG. 2. The flat surface of the gel or resin is coplanar with one surface of the U-shaped cut-out. The ASIC 205 serves the same functions as the ASIC 105 of FIG. 1 and communicates with an identical processor to the one described with reference to FIG. 1.

FIG. 2 shows a finger 203 located between the signal acquisition component 201 and a mechanism for applying force 202. The finger is located with the nail adjacent the mechanism 202 and the bottom of the finger adjacent the flat surface. A soft pad 204 for transmitting the force from the mechanism 202 to the finger 203 is mounted on the mechanism 202.

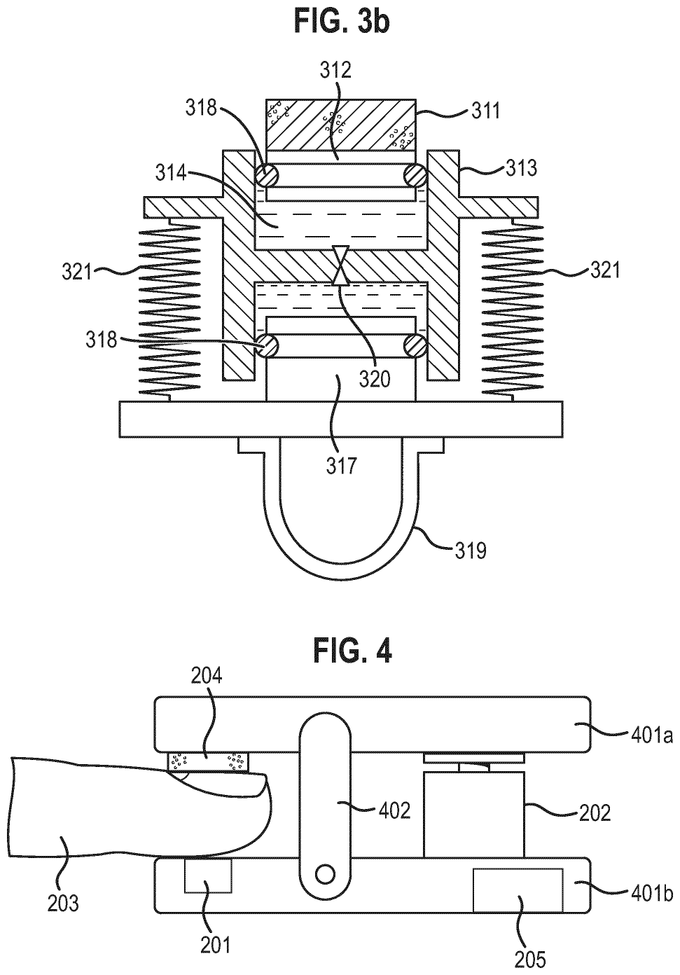

In FIG. 3a, there is a soft pad 302 resting on a flexible-walled vessel 301. The flexible-walled vessel can be made of a stiff rubber-like material resembling a squash ball. A valve 304 allows air to escape from the vessel slowly and to flow back in quickly. The user presses an actuator 303 that compresses the rubber. The actuator is held in the compressed position by a latch (not shown). The pressure in the vessel causes the soft pad to press hard on the finger. As the air leaks out through the valve, the pressure in the vessel falls and the force on the soft pad also falls. After the measurement is completed, the latch (not shown) and the air flows back into the vessel, ready to be used again.

FIG. 3b shows a more complex mechanism that has a cylinder 313 divided by a barrier that carries a valve 320. A piston 312 carries a soft pad 311 for pressing against the finger. The cylinder contains a fluid 314 which may be a gas or a liquid. An actuator 317 compresses the fluid by the action of one or more springs 321. The valve allows the fluid to flow quickly from the region of the cylinder under the piston to the region above the actuator, but only slowly in the opposite direction. The actuator and piston are sealed to the cylinder with O-rings 318. In operation, the user pulls the handle 319 downwards, causing fluid to flow easily from the upper to the lower part of the cylinder, causing the actuator to move down and causing the spring or springs to be extended. The user then releases the handle and the force generated by the springs causes fluid to flow through the valve and thus causes the piston to rise and press the soft pad against the body part. As more fluid passes through the valve, the pressure on the piston continues to rise and steadily increases the force applied to the soft pad.

It will be obvious to a person skilled in the art that many variations of these force-generating means are possible. For example, the O-rings might leak so the two regions of the cylinder might alternatively contain sealed bellows.

In both FIG. 3a and FIG. 3b, the varying force of the soft pad on the finger causes a varying pressure in the body part and that varying pressure in the finger 203 is measured by the pressure sensor of the component 201.

The ASIC 205 collects, digitises and communicates the measured signals to the processing means which processes the signals to provide a measure of the blood pressure in the finger 203.

FIG. 4 shows a second device according to the present invention. The items 201, 202, 203, 204 and 205 are identical to those items as described for FIG. 2. However, they are arranged differently as shown in FIG. 3. The device of FIG. 3 comprises two arms 401a and 401b. The two arms are linked at their centres by a hinge 402. Items 201 and 205 are located in opposite ends of arm 401a. The soft pad 204 is mounted on the end of the arm 401b opposite the signal acquisition component 201. The mechanism for applying force 202 is located between the ends of the arms 401a, 401b remote from the signal acquisition component 201 and soft pad 204.

In use, the user actuates the mechanism 202 and places the finger 203 between the soft pad 204 and the signal acquisition component 201. As mechanism operates, the left hand ends (as seen in FIG. 4) of the arms 401a, 401b are pivoted towards each other by the mechanism, thus applying varying force to the finger 203. The signals produced by the pressure sensor and the photodetectors are then processed as for FIG. 2.

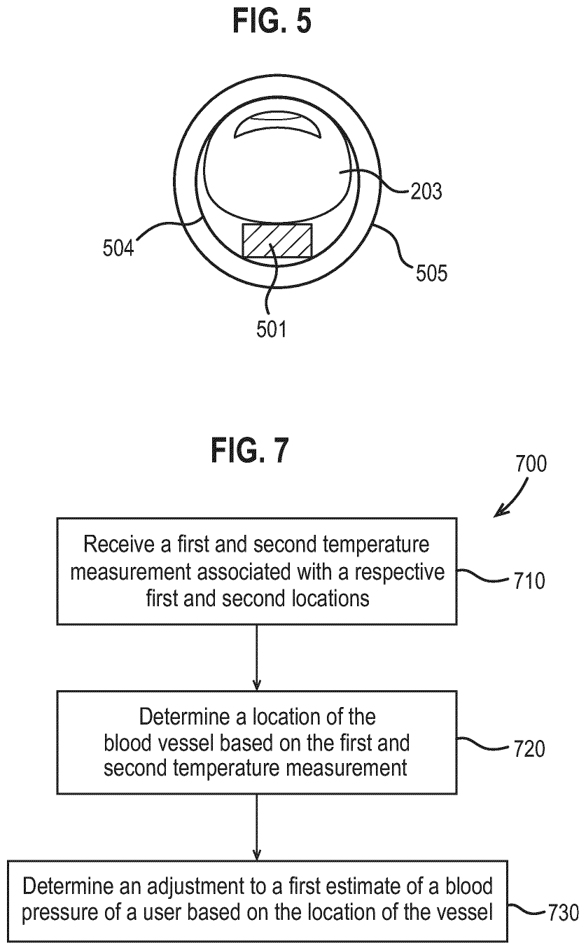

A third device according to the invention is shown in FIG. 5. In this device, a signal acquisition component 501 as described with reference to FIG. 1 is used. The component 501 is located within an inflatable cuff having an inner wall 504 and outer wall 505. In use, a finger 203 is placed with its lower part on the flat surface of the component 501. The cuff is then inflated to apply varying force to the finger 203 which produces varying pressure in the finger 203. Signal processing is carried out as described for FIG. 2.

### 2. Second Aspect

The core body temperature is an important parameter related to the health of a user that is typically approximated by the temperature of a blood vessel. Ideally, to measure core body temperature, a measurement is taken of the blood vessel's temperature in the vessel, but such a measurement is challenging to achieve in a non-invasive manner.

Under some circumstances, the accuracy of a temperature measurement of the core body temperature of a user may be improved by using more than one temperature sensor so that several different points on the surface of the skin are sensed. Using multiple temperature sensors allows for a blood vessel to not be directly under any specific sensor and also allows the temperature of the skin above the blood vessel to be compared with that of the skin that is not above the blood vessel, ultimately allowing an improved estimate of the temperature of the arterial blood and reduced error due to user positioning of the sensors.

Further, a cooling effect between the arterial blood and the surface of the skin exists at heat propagates through tissue and skin. The effect is minimised if the thickness of the skin and tissue between the blood vessel and the surface is minimised. Ideally the temperature is measured directly above the blood vessel, where the cover is thinnest. It is preferable that the measurement also provides an indication of the cooling due to ambient conductions.

It may be difficult for a user to consistently place the appropriate sensors directly above the blood vessel and the precise location of a given blood vessel can vary from user to user. Therefore, it is necessary to compensate firstly for misalignment of the sensors over the blood vessel and secondly for the variation in temperature from the skin to the blood in the blood vessel.

The second aspect is directed towards an improved method of determining a parameter related to the health of a user. Specifically, this may be measuring the core body temperature of a patient itself or improving the estimate of a patient's blood pressure based on the measurements from an array of temperature sensors.

The present invention also relates to a method of determining the core body temperature of a patient based on temperature measurements from at least two temperature sensors. The temperature sensors may be either contact devices, that measure the temperature of the skin by touching, or radiative devices, that measure the temperature of the skin by detecting infra-red radiation. The temperature sensors may be arranged in an array with a fixed separation, or they may be independently placeable. Preferably, the temperature sensors are arranged as close to a blood vessel as possible.

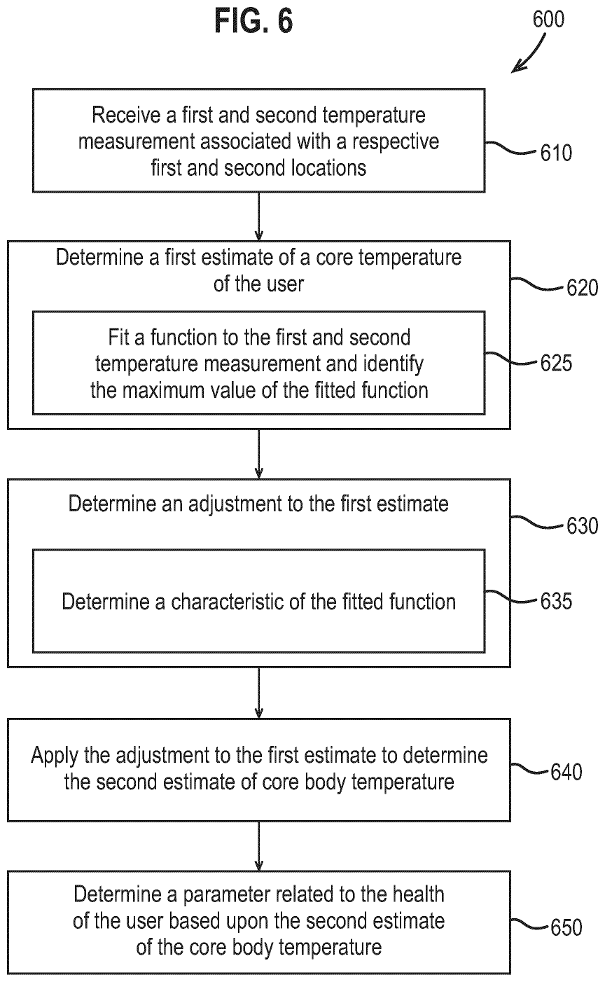

FIG. 6 shows an exemplary method of determining a parameter related to the health of a user according to the invention. In step 610 a first temperature measurement is received by a processor from a first temperature sensor. The first temperature sensor is associated with a first location on the surface of the body of the user. A second temperature measurement is also received by a processor from a second temperature sensor. The second temperature sensor is associated with a second location on the body.

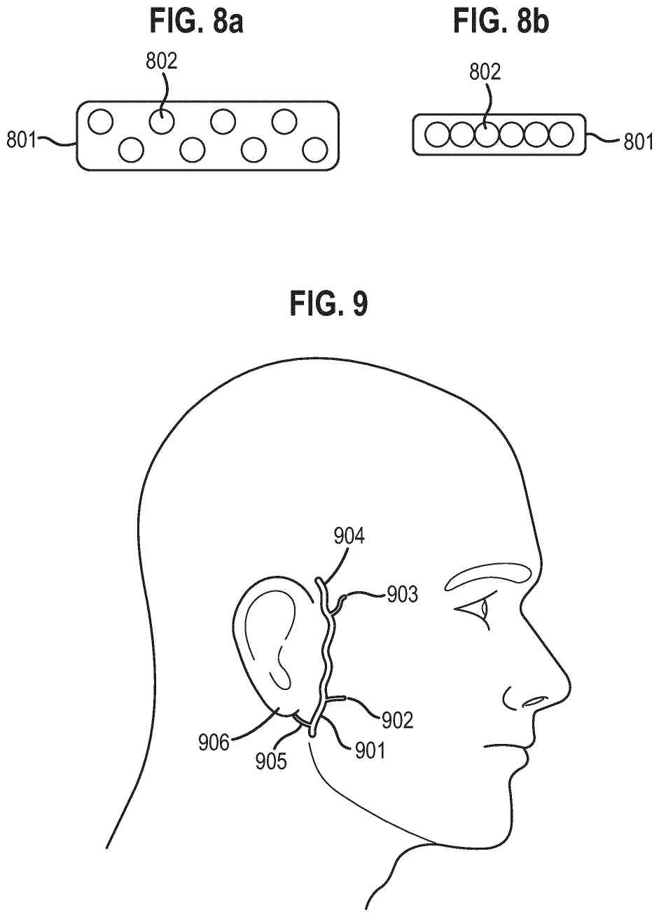

FIG. 8 shows two possible embodiments of the array of temperature sensors for receiving the first and second temperature measurements. Other embodiments or arrangements of the sensors are possible. In FIG. 8 there is a housing 801 that carries an array of surface temperature sensors 802. In FIG. 8a there is shown eight temperature sensors, arranged in a grid, and in FIG. 8b there is shown six temperature sensors, arranged in a line.

In step 620 a first estimate of the core body temperature is determined based on the first temperature measurement. Since the core body temperature (as represented by the blood vessel temperature) is generally higher than the surface temperature, this will lead to the temperature of the skin directly above the blood vessel to be warmer and closer to the real core body temperature.

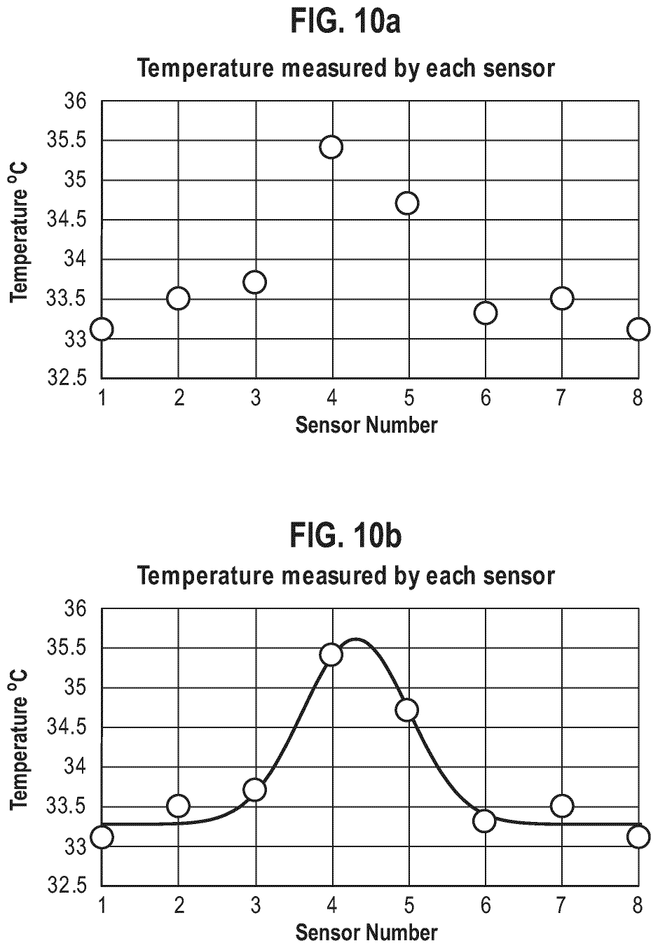

The temperature measured by each sensor will depend on its distance from the blood vessel. FIG. 10a shows the typical distribution of measured temperatures for a linear array of 8 sensors. Based on the temperature of the skin above the blood vessel being warmer, the blood vessel is located between sensor 4 and 5 and closer to sensor 4. It is apparent that the temperature measured by sensor 4 (35.4° C. in the example of FIG. 10) is a better estimate of the core body temperature than that of one of the sensors further from the blood vessel. However, because the blood vessel is not located exactly under sensor 4, it has still underestimated the peak temperature on the surface of the skin.

Returning to FIG. 6, step 620, in the specific example, additionally comprises step 625 which comprises fitting a function to the first and second temperature measurements. A first estimate of the core body temperature may then be determined from a property of the fitted function such as the peak or maximum of the fitted function. Fitting a function to the data enables the temperature measurements to be interpolated across the body part (the cheek in this example) as well as determine the peak temperature on the surface of the skin at that location.

The fitted function may be a Gaussian function. FIG. 10b shows the measured data from FIG. 10a with the plot of a fitted Gaussian function found by regression to the data. Other mathematical formulas may be used, such as one that takes account of the underlying physics and physiology. The fitted curve has a peak of 35.6° C. located approximately ⅓ of the distance between sensors 4 and 5. Hence, an improved estimate of the temperature of the skin directly over the blood vessel is obtained. As mentioned previously, core body temperature is generally higher than the temperature of the skin. However, the difference between these two values will be smallest in the skin above the blood vessel. Thus, the first estimate of the core body temperature may be the maximum value of the skin temperature as found from the fitted function.

Since core body temperature is generally higher than the temperature of the skin, to improve the first estimate it is necessary to understand the cooling between the blood vessel and the skin so that the estimate can be adjusted and improved accordingly. Returning to FIG. 6, in step 630 of method 600, an adjustment to the first estimate is determined based on the first temperature measurement and the second temperature measurement.

Step 630 may further comprise step 635 where the adjustment is calculated based on a characteristic of the fitted function. The adjustment may be a function of the variance of the fitted gaussian function shown in FIG. 10b. Of course, it will be appreciated that, should the fitted function be a Gaussian, the parameters such as variance represent the spatial spread across the measurement locations rather than statistical properties. The shape of the fitted function contains information on the cooling due to ambient conditions. For example, if there is less cooling, the gradient and/or difference between adjacent sensors will be reduced because the conduction through the tissue will be relatively more effective.

Because it is difficult analytically to model the dependence between the characteristic of the fitted function (such as the variance of the fitted Gaussian) and the variation of temperature from the blood inside the blood vessel to the surface of the skin above it, a machine learning technique may be used to derive a law relating the characteristics of the fitted curve, such as its width, to the temperature difference. For example, the adjustment may be inversely proportional to the variance of the fitted Gaussian function.

In step 640 of method 600, the adjustment is applied to the to the first estimate of the core body temperature to then determine a second estimate of core body temperature. The second estimate of core body temperature is then used to determine the parameter related to the health of the user in step 650. The parameter related to the health of the user may be the core body temperature, or it may be an alternative parameter such as the user's blood pressure.

There are many variations to method 600 which may be implemented to preserve computing resources or reduce device size. As will be explained further below, the temperature sensors may be integrated into a garment or wearable.

While commonly three measurements are required to fit a curve such as a Gaussian, it is possible to fit a curve with two temperature measurements using a Bayesian approach or with an approach that allows additional constraints to be considered. For example, if the location of one of the sensors is known relative to the facial structure (e.g. it is known that one of the at least two temperature sensors is against the ear) information such as the typical distance from the known location to the blood vessel can be used to inform the fitting of the function. Additionally, or alternatively, the information related to the typical variation between the skin and blood temperature can also be used to bound the peak of the Gaussian function. A Bayesian approach may also be applied when there are more than two measurements, and such additional constraints and prior knowledge may be used to minimise the impact of measurement noise.

Nonetheless, there may be further temperature measurements. For example, the at least two temperature sensors may be at least three temperature sensors and a third temperature measurement is received from a third temperature sensors of the at least three temperature sensors where the third temperature sensors may be associated with a third location on the body. The third or further temperature measurements may also be used in further refining any of the determinations made by method 600 such as the first or second estimates or the adjustment.

When there are three or more measurements the mathematical procedure may be adapted to be used for different dispositions of the temperature sensors, including non-uniform distributions.

In step 625 of method 600, rather than identifying the peak or maximum of the fitted function, other properties could be used depending on the function. For example, the value of the function where the area under the function up to that point is half the total area i.e. a median of the function. Alternatively, the point at which the gradient of the function is zero. In the case where a step function or other discontinuous function is used, the point halfway along the step in the spatial direction may be used.

As an alternative to step 625, the first estimate of core body temperature may be the highest temperature measurement received from the at least two temperature sensors or the measurement that indicates the highest core body temperature. In such an alternative, step 635 may also be modified to instead calculate the adjustment based on a comparison or difference between the first temperature measurement and the second temperature measurement. Additionally, or alternatively, the adjustment may be the difference between the first estimate and the peak of a function fitted to the first temperature measurement and the second temperature measurement in a similar fashion as described above in connection with step 625 of method 600. These alternative steps may be suitable for processors where processing power is limited, or for improving the speed of calculating the estimates.

The first and second locations may be on the surface of the body in the same region as a blood vessel of interest such as a vein or artery. For example, the temperature sensors may be associated with a region of the cheek in which the external carotid artery is located (as shown in FIG. 9). The at least two sensors may be arranged so that it is approximately perpendicular to the line of the artery. In practice, for a user sitting or standing, the array is positioned so that the temperature sensors are aligned horizontally. The temperature sensors may also be placed above the temporal artery on the forehead and aligned in a similar manner perpendicular to the line of the artery.

For illustration, consider the carotid artery in front of the ear, although this invention is not restricted to this artery or region of the body. FIG. 9 shows its typical location. The ear lobe 906 is at the bottom left. The external carotid artery 901 runs up from the neck. It has branches: the posterior auricular artery 905, the maxillary artery 902 and the transverse facial artery 903, and then becomes the superficial temporal artery 904. It would be convenient to measure on the skin in front of the ear, but the exact position of the artery varies between people.

The temperature sensors should be within a distance of one another such that any variation from the temperature of the skin can be significantly attributed to the presence of the blood vessel nearby. For example, the separation between each of the locations measured by the at least two temperature sensors may be less than 30 cm, less than 15 cm, less than 5 cm, less than 2 cm, or less than 1 cm. Alternatively the sensors, and their corresponding locations, may be tightly-packed as shown in FIG. 8.

The parameter related to the health of the user may be the core body temperature itself or some other parameter such as blood pressure of the user or the oxygenation of the blood of the user. For example, when a function is fitted to the temperature measurements, the peak of the measurements corresponds to a more accurate estimate of the core body temperature than the raw measurements from the at least two temperature sensors, as discussed previously. In addition, the location of the peak in the fitted function can also indicate the location of the blood vessel relative to the device. This location can be taken into account when calculating other measurements such as calculating the user's blood pressure since the distance from any blood pressure sensors to the blood vessel can then be determined, and the reduction in pressure variation based on this distance.

FIG. 7 shows an additional exemplary method 700 of determining a parameter related to the health of a used according to the invention. Method 700 may also be used in assisting the determination of a parameter related to the health of the user. Method 700 is similar to method 600 except that the temperature sensor measurements are used to assist the calculation of a blood pressure measurement. Step 710 of method 700 is substantially similar to step 610 of method 600, as previously described. Step 720 is similar to step 620, in that the location of the blood vessel is determined by fitting a function to the first temperature measurement and second temperature measurement. The location of the blood vessel may then be determined based on a characteristic of the fitted function such as the location of the peak or the maximum of the function. In step 730 an adjustment to a first blood pressure measurement may be calculated based on the location of the blood vessel as determined by step 720. The adjustment may be a function of the relative location of the blood vessel and the location where the blood pressure is measured from, with the adjustment being larger the further from the blood vessel that location lies. Alternatively, the adjustment may be based on the variance of the fitted function in a similar manner to that described with respect to method 600.

The location of the blood vessel may be known relative to the temperature sensors, blood pressure sensors or some other point from which the processor may receive signals.

Optionally, method 700 may be extended to apply the adjustment to the first blood pressure measurement to determine a second estimate of the blood pressure of the user. This second estimate of the blood pressure of the user may then be used to determine the parameter related to the health of the user.

It is possible to swap the primary measurement sensors of method 600 and 700. For example, the blood pressure measurements could be used to determine the location of the blood vessel and used to determine an adjustment to an estimate of core body temperature. Alternatively, the blood pressure can be determined from at least two pressure sensors, receiving a first pressure measurement and second pressure measurement in a fashion similar to that of the temperature measurements of method 600. A first estimate of the user's blood pressure can then be determined from the first pressure measurement. An adjustment to the first blood pressure estimate can then be calculated based on the first and second pressure measurements (such as fitting a Gaussian or taking the highest value as previously described in connection with method 600) and finally a parameter related to the health of the user can be based upon a second estimate of the user's blood pressure which is determined by applying the adjustment to the first estimate of the user's blood pressure.

Both method 600 and 700 may also include the step of providing the determined health parameter to the user. The parameter may be provided visually or audibly.

It is possible that methods 600 and 700 may be executed in parallel or at the same time. Since both methods require at least two temperature measurements from at least two sensors associated with two locations, and a function is fit to the at least two temperature measurements, the same fitted function and measurements can be used across both methods. The remaining steps of each method may then be executed in parallel, sequentially or by alternating steps. Other determinations in each of the methods, such as calculating the adjustment may also be shared between the methods.

A computer-readable medium may be configured to store instructions that, when executed by a processor, cause the processor to carry out the methods described in the present application. The computer readable medium may be ROM, RAM, a USB stick, hard-drive, CD, floppy disk or other means of storing computer-executable instructions.

A processor may be configured to carry out the methods described in the present application. A single processor may be configured to carry out all of the methods described in the present application. Such a processor may be for a personal health monitor (PHM) or it may be configured to operate as a part of the device of the first aspect.

The personal health monitor includes a signal acquisition device (SAD) which comprises at least two sensors including a first sensors and a second sensor. These sensors may be the at least two temperature sensors of methods 600 or 700, or they may be other sensors to implement variants of these methods such as the pressure sensors previously discussed. There may also be additional sensors capable of measuring the same property (i.e. temperature, pressure) that can be used to further improve the fitting of a function for the first estimate, as outlined previously.

The at least two sensors may be fixed within a housing of the SAD such that moving of the housing causes movement of each of the at least two sensors simultaneously. The housing also maintains a fixed separation between the sensors. The arrangement of sensors in the housing may be similar or identical to that as discussed previously in connection with FIG. 8.

The housing may comprise a contact surface such that the at least two sensors can be pressed against the surface of the body and allow the first and second sensors to be associated with the first and second locations. Alternatively, in the case of sensors that do not require contact such as radiative temperature sensors, it may not be necessary to include a contact surface.

When present, the contact surface of the housing of the SAD may be configured to conform or match the shape of the body part such that every sensor in the housing can be simultaneously in contact the body. For example, the contact surface may be made of a flexible material such that when it is pressed against the cheek, the contact surface will flex to the shape of the cheek such that more of the contact surface can contact the cheek than would be possible if the contact surface did not flex. Alternatively, the contact surface may be shaped such that the skin is deformed around the contact surface. This alternative can help maintain the blood vessel in a specific location and minimise its movement between different measurements.

The PHM may be a personal hand-held monitor (PHHM). Such a PHHM may be a mobile phone or other hand-held device adapted to include the SAD. Alternatively, the PHM may wirelessly connect and receive measurement signals for processing from the SAD such as over Bluetooth, wi-fi, radio or other form of network connectivity.

The SAD may be disposed within a garment or wearable device. For example, a hat, pair of headphones or smart glasses may have the SAD configured such that when the garment is worn, the SAD is pressed against the cheek over the carotid artery. Other possible garments that the SAD may be disposed within could include a helmet, a cap, a piece of safety equipment an earphone or pair of earphones, a virtual reality headset or piece of clothing.

The garments or wearables can be any piece of clothing or piece of technology that can be worn or attached to the user and may be clothes, jewellery, safety equipment, glasses, belts, straps or shoes.

A pair of gloves may include the at least two sensors around the finger such that, when pressed against the finger, appropriate measurements can be made. Other garments that also cover blood vessels of interest may have a SAD integrated into them. The garments may also have the processor integrated within them.

The scope of the invention is not limited by the above examples but is determined by the following claims.

1-51. (canceled)

52. A device for measuring blood pressure in a body part comprising:
a signal acquisition component comprising:
a flexible and essentially incompressible gel or resin comprising a surface that is adapted to contact the body part;
a pressure sensor embedded in the gel or resin;
one or more photoemitters for emitting light into the body part when the body part is in contact with the surface of the gel or resin;
one or more photodetectors for detecting light scattered by or transmitted through the body part;
a mechanism for applying force, wherein the mechanism for applying force is adapted to apply varied force to the body part in a direction towards the surface of the gel or resin; and
a processing means that is programmed to analyse signals from the pressure sensor and the one or more photodetectors to provide a measurement of the blood pressure in the body part.

53. The device of claim 52, wherein the mechanism for applying force is arranged such that, in use, the force is applied indirectly to the body part through a hinged mechanism or a lever.

54. The device of claim 53, wherein the hinged mechanism comprises:
a first arm, wherein the surface of the gel or resin and the mechanism for applying force are located on opposite ends of the first arm;
a second arm; and
a hinge linking the first arm and the second arm.

55. The device of claim 52, wherein the mechanism for applying force is arranged such that, in use, the force is applied directly to the body part.

56. The device of claim 55, further comprising a soft pad that is located, in use, between the mechanism for applying force and the body part.

57. The device of claim 56, wherein the body part is a finger, wherein the soft pad is positioned adjacent to a nail of the finger.

58. The device of claim 55, wherein the mechanism for applying force comprises:
a flexible-walled vessel comprising a valve;
an actuator configured to compress the flexible-walled vessel; and
a latch configured to hold the actuator in the compressed position.

59. The device of claim 55, wherein the mechanism for applying force comprises a piston and a cylinder.

60. The device of claim 59, wherein the mechanism for applying force further comprises:
one or more springs; and
an actuator configured to compress a fluid in the cylinder by the action of the one or more springs.

61. The device of claim 60, wherein the cylinder comprises:
a piston region that corresponds to the piston,
an actuator region that corresponds to the actuator, and
a valve positioned between the piston region and the actuator region, wherein the valve allows fluid to flow more quickly from the piston region to the actuator region than from the actuator region to the piston region.

62. The device of claim 61, wherein the mechanism for applying force comprises a clockwork mechanism that is preloaded by the user.

63. The device of claim 52, wherein the mechanism for applying force is adapted to generate force which increases or decreases monotonically after the mechanism for applying force is activated by the user.

64. The device of claim 63, wherein the processing means controls the rate of change for the force that increases or decreases monotonically.

65. The device of claim 64, wherein the processing means controls the rate of change by operating a valve that determines the rate of flow of a fluid being forced through a small aperture.

66. The device of claim 63, wherein a dash pot controls the rate of change for the force that increases or decreases monotonically.

67. The device of claim 63, wherein an air damper controls the rate of change for the force that increases or decreases monotonically, wherein the air damper comprises a spinning vane that is geared to spin as the mechanism for applying force advances.

68. The device of claim 52, wherein the processing means is adapted to control the variation of the force applied, in use, to the body part by the mechanism for applying force.
